(19) United States
(12) Patent Application Publication
(10) Pub. No.: US 2024/0146084 A1
(43) Pub. Date: May 2, 2024

(54) CIRCUIT, METHOD, AND OXIMETER FOR INDICATING POWER SUPPLY BY APPLYING DOUBLE-COLOR LIGHT-EMITTING DIODE

(71) Applicant: Guangdong Genial Technology Co., Ltd., Zhaoqing (CN)

(72) Inventor: Chang SHU, Zhaoqing (CN)

(21) Appl. No.: 18/140,820

(22) Filed: Apr. 28, 2023

Related U.S. Application Data
(63) Continuation of application No. PCT/CN2020/124295, filed on Oct. 28, 2020.

(51) Int. Cl. H02J 7/00; A61B 5/00; A61B 5/1455

(52) U.S. Cl. CPC H02J 7/0049; A61B 5/14552; A61B 5/6826; A61B 5/6838

(57) ABSTRACT

A circuit, method, and oximeter for indicating a power supply by applying a double-color light-emitting diode (LED) are provided. The circuit, applied to a finger clip type pulse oximeter, comprises the following components: an MCU module U1, a double-color LED D1 connected to the MCU module U1, a voltage measurement module connected to the MCU module U1, a rechargeable battery module connected to the voltage measurement module, and a power supply input module that is suitable for connection with an external power supply and is connected to the MCU module U1 and the rechargeable battery module, wherein the MCU module U1 is suitable for controlling the double-color LED D1 according to the states of the power supply input module and the rechargeable battery module, so that the finger clip type pulse oximeter presents a visual result directly observable by the human eyes. The double-color LED is configured for the finger clip type pulse oximeter having a small display screen, so that a user can know about the power supply condition of the finger clip type pulse oximeter more intuitively.

### TECHNICAL FIELD

The present invention relates to a circuit, method, and oximeter for indicating a power supply by applying a double-color light-emitting diode (LED).

### BACKGROUND

The existing finger clip type pulse oximeters are mainly powered by AAA7 batteries, and cannot be powered by lithium batteries and the like; besides, when the battery level, represented by an energy bar, is low, a prompt that the LCD screen flashes or the energy bar is empty is usually given.

However, since the LCD screen of the finger clip type pulse oximeter is generally about 1 inch in size, the display icon for the energy bar is small; moreover, different from using a mobile phone, a user, when using a finger clip type pulse oximeter, generally does not pay close attention to the battery level, and may not know that there is no electricity only until the battery is completely dead or the finger clip type pulse oximeter cannot be turned on, unless there is a more intuitive way to observe.

Therefore, the existing finger clip type pulse oximeters have the following disadvantages:
(1) Displaying the low battery level through the flashing of the LCD screen is easy to cause trouble for a user and interfere with viewing the test data;
(2) although the energy bar can display the battery level, a user tends to neglect the display here when using the oximeter, such that the user may not know the battery is dead only until the oximeter is shut down automatically; and
(3) failure to support the online charging function may cause the oximeter to become unusable because the battery is not replaced in time, and especially may also increase a user's psychological pressure of using the oximeter if there is no spare battery at home.

### SUMMARY

A first object of the present invention is to provide a circuit for indicating a power supply by applying a double-color LED, this circuit enabling a user to more intuitively know the battery level of the finger clip type pulse oximeter through the double-color LED.

A second object of the present invention is to provide a method for indicating a power supply by applying the above circuit, this method enabling a user to more intuitively know the battery level of the finger clip type pulse oximeter.

A third object of the present invention is to provide an oximeter for indicating a power supply by applying a double-color LED, this oximeter enabling a user to more intuitively know its battery level.

For the above-mentioned first object, the present invention adopts following technical solution:

A circuit, applied to a finger clip type pulse oximeter, for indicating a power supply by applying a double-color LED is provided, comprising the following components:
an MCU module U1;
a double-color LED module, including a double-color LED D1 and connected to the MCU module U1;
a voltage measurement module connected to the MCU module U1;
a rechargeable battery module connected to the voltage measurement module; and
a power supply input module, which is suitable for connection with an external power supply and is connected to the MCU module U1 and the rechargeable battery module;
wherein the MCU module U1 is suitable for controlling the double-color LED D1 according to the states of the power supply input module and the rechargeable battery module, so that the finger clip type pulse oximeter presents a visual result directly observable by the human eyes.

Furthermore, with the power supply input module including a socket U2, a fuse F2 is connected between the socket U2 and the output terminal of the power supply input module; the output terminal of the power supply input module is connected to the rechargeable battery module, and connected to the MCU module U1 through an insertion detection module.

Furthermore, the insertion detection module includes a resistor R1, which has one terminal connected to the MCU module U1 and the other terminal connected to the output terminal of the power supply input module.

Furthermore, the rechargeable battery module comprises a lithium battery BAT1, and a charging management chip U3 connected to the output terminal of the power supply input module; the lithium battery BAT1, with its negative electrode grounded, has its positive electrode connected to the charging management chip U3 and connected to the MCU module U1 through the voltage measurement module.

Furthermore, the voltage measurement module includes resistors R7 and R8, one terminal of the resistor R7 being connected to the positive electrode of the lithium battery BAT1; the resistor R8 has its one terminal grounded, and the other terminal connected to the other terminal of the resistor R7 and then connected to the MCU module U1.

Furthermore, the double-color LED D1 comprises a first LED capable of exhibiting a first color, and a second LED capable of exhibiting a second color, with the first color different from the second color; the double-color LED module further comprises resistors R4 and R5; the anode of the first LED is connected to a VDD pin of the MCU module U1 through the resistor R4, and the anode of the second LED is connected to the same VDD pin of the MCU module U1 through the resistor R5; the cathode of the first LED is directly connected to one I/O pin of the MCU module U1, and the cathode of the second LED is directly connected to the other I/O pin of the MCU module U1.

Furthermore, the above description that “the finger clip type pulse oximeter presents a visual result directly observable by the human eyes” is specifically as follows:

When the power supply input module is connected to the external power supply, and the lithium battery BAT1 is being charged, the MCU module U1 controls the first LED to flash in the first color at a predetermined frequency;
when the power supply input module is connected to the external power supply, and the lithium battery BAT1 is fully charged, the MCU module U1 controls the first LED to be always on in the first color;
when the power supply input module is not connected to the external power supply, and the MCU module U1 detects that the voltage of the detection terminal of the voltage measurement module is lower than a first predetermined voltage, the MCU module U1 controls the second LED to be always on in the second color; and
when the power supply input module is not connected to the external power supply, and the MCU module U1 detects that the voltage of the detection terminal of the voltage measurement module is lower than a second predetermined voltage, the MCU module U1 controls the second LED to flash in the second color at a predetermined frequency;
wherein the second predetermined voltage is lower than the first predetermined voltage.

Furthermore, the charging management chip has a VIN pin connected to the power supply input module, a BAT pin connected to the lithium battery BAT1 for charging current output, and a CHG pin connected to an indication LED D2 for charging state output, the indication LED D2 capable of exhibiting a third color.

For the above-mentioned second object, the present invention adopts following technical solution:

When the power supply input module is connected to the external power supply, and the lithium battery BAT1 of the rechargeable battery module is being charged, the MCU module U1 controls the first LED of the double-color LED D1 to flash in the first color at a predetermined frequency;
when the power supply input module is connected to the external power supply, and the lithium battery BAT1 of the rechargeable battery module is fully charged, the MCU module U1 controls the first LED of the double-color LED D1 to be always on in the first color;
when the power supply input module is not connected to the external power supply, and the MCU module U1 detects that the voltage of the detection terminal of the voltage measurement module is lower than a first predetermined voltage, the MCU module U1 controls the second LED of the double-color LED D1 to be always on in the second color; and
when the power supply input module is not connected to the external power supply, and the MCU module U1 detects that the voltage of the detection terminal of the voltage measurement module is lower than a second predetermined voltage, the MCU module U1 controls the second LED of the double-color LED D1 to flash in the second color at a predetermined frequency;
wherein the second predetermined voltage is lower than the first predetermined voltage.

For the above-mentioned third object, the present invention adopts following technical solution:

An oximeter, being of a finger clip type pulse oximeter, for indicating a power supply by applying a double-color LED is provided, comprising a body and the above-mentioned circuit; the body is provided on the surface with a display screen and an indication position located on one side of the display screen, with the double-color LED D1 set corresponding to the indication position.

The present invention has the following beneficial effects: The double-color LED is configured for the finger clip type pulse oximeter having a small display screen, so that two colors of light can be emitted by the double-color LED and can be controlled through the power supply input module and the rechargeable battery module, enabling the finger clip type pulse oximeter to present a visual result directly observable by the human eyes; in this way, through the intuitive lighting display of the LED, a user can visually check the battery level, thereby eliminating the user's psychological pressure of being unable to use the oximeter due to a low battery level.

### BRIEF DESCRIPTION OF DRAWINGS

FIG. 1 schematically shows the structure of a finger clip type pulse oximeter according to an example of the present invention;

FIG. 2 is a block diagram showing the circuit structure of a finger clip type pulse oximeter according to an example of the present invention;

FIG. 3 shows the circuit structure of an MCU module according to an example of the present invention;

FIG. 4 shows the circuit structure of a voltage measurement module according to an example of the present invention;

FIG. 5 shows the circuit structure of a rechargeable battery module according to an example of the present invention;

FIG. 6 shows the circuit structure of a power supply input module according to an example of the present invention;

FIG. 7 shows the circuit structure of an insertion detection module according to an example of the present invention; and

FIG. 8 shows the circuit structure of a double-color LED module according to an example of the present invention.

### DETAILED DESCRIPTION OF EMBODIMENTS

In order to facilitate a better understanding of the objects, structures, features and effects of the present invention, the present invention will now be further described with reference to the accompanying drawings and specific embodiments.

An example of the present invention provides an oximeter for indicating a power supply by applying a double-color LED, with the oximeter being of a finger clip type pulse oximeter.

As shown in FIG. 1, the oximeter comprises a body 4 and a circuit, the circuit being arranged in the body 4 and including a rechargeable lithium battery; the body 4 is provided on the surface with a display screen 3, on one side of which is arranged a touch button 2 and two spaced indication positions; indicator lights 1 and 5 are respectively arranged at the two indication positions, wherein the indicator light 5 is a double-color LED for indicating the power supply, and the indicator light 1 is used for indicating other states; on the other side of the display screen is further arranged a socket 6 suitable for charging access, which is, in an example, a USB socket connected to an external power supply through a charging cable for charging the lithium battery.

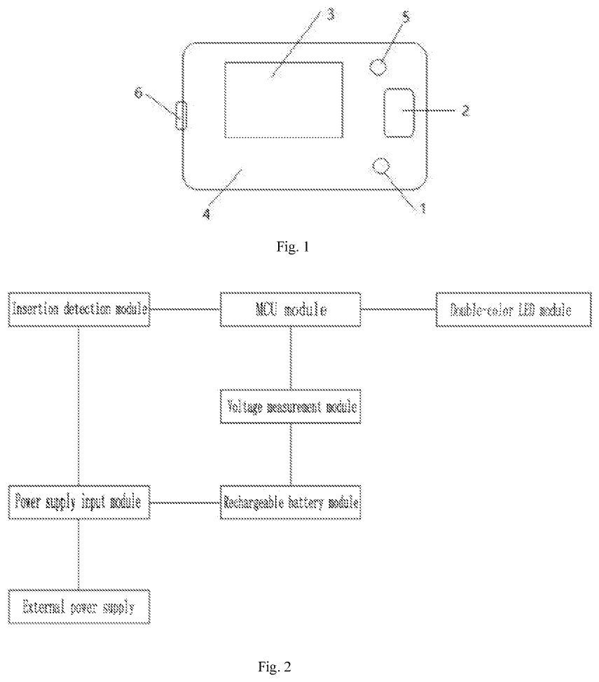

The circuit therein uses a double-color LED to indicate the power supply. As shown in FIG. 2, the circuit comprises an MCU module, a double-color LED module connected to the MCU module, a voltage measurement module connected to the MCU module, a rechargeable battery module connected to the voltage measurement module, and a power supply input module that is suitable for connection with an external power supply and is connected to the MCU module and the rechargeable battery module.

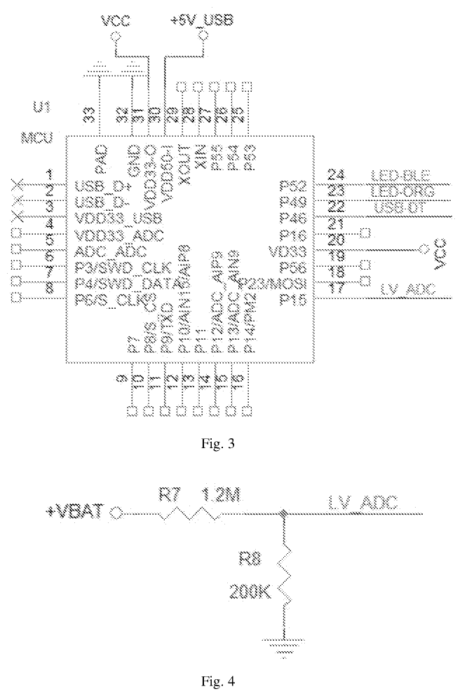

An MCU (i.e. micro-control unit) module of any suitable model, e.g. STM32L071CB, can be used in the present invention. In an example, the pin structure and circuit connection relationship of the MCU module U1 are shown in FIG. 3.

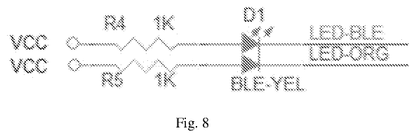

As shown in FIG. 8, the double-color LED module comprises a double-color LED D1, a resistor R4, and a resistor R5; the double-color LED D1 comprises a first LED capable of exhibiting a first color, and a second LED capable of exhibiting a second color, with the first color different from the second color. The first and second colors may be any different colors; for example, in an example, the first color is blue and the second color is orange. The anode of the first LED is connected to the MCU module U1 through the resistor R4, and the anode of the second LED is connected to the MCU module U1 through the resistor R5. In an example, the resistors R4 and R5 each have a resistance of 1 kΩ. The cathode of the first LED is directly connected to one I/O pin P52 of the MCU module U1, and the cathode of the second LED is directly connected to the other I/O pin P49 of the MCU module U1.

The MCU module U1 is suitable for controlling the double-color LED according to the states of the power supply input module and the rechargeable battery module, so that the finger clip type pulse oximeter presents a visual result directly observable by the human eyes.

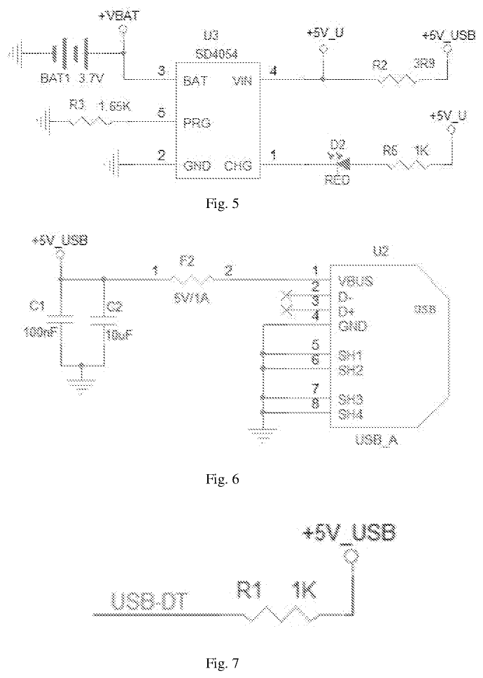

As shown in FIG. 5, the rechargeable battery module comprises a lithium battery BAT1, and a charging management chip U3 connected to the output terminal +5V_USB of the power supply input module; the lithium battery BAT1, with its negative electrode grounded, has its positive electrode +VBAT connected to the charging management chip U3 and connected to pin P15 of the MCU module U1 through the voltage measurement module. The charging management chip may be of any suitable model, e.g. SD4054. In an example, the charging management chip U3 has a VIN pin connected to the power supply input module, a BAT pin connected to the lithium battery BAT1 for charging current output, a PRG pin for charging current setting, charging current monitoring and shutdown, a GND pin for grounding, and a CHG pin for charging state output. The CHG pin is connected to an indication LED D2, which is capable of exhibiting a third color. The third color may be different from both the first color and the second color, for example, the third color may be yellow. The indication LED D2 is used to detect the working state of the charging management chip. In some examples, as shown in FIG. 1, the indication LED D2 can be correspondingly arranged at the other indication position of the body 4; that is, one indicator light 5 on the surface of the body 4 is a double-color LED D1, and the other indicator light 1 is an indication LED D1. In this way, whether the charging management chip U3 is working normally can be judged by directly observing the indication LED D2 with the human eyes.

As shown in FIG. 4, the voltage measurement module includes resistors R7 and R8, one terminal of the resistor R7 being connected to the positive electrode +VBAT of the lithium battery BAT1; one terminal of the resistor R8 is grounded, and the other terminal is connected to the other terminal of the resistor R7 to form a detection terminal LV_ADC, which is connected to pin P15 of the MCU module U1. In an example, the resistance of R7 is 1.2 MΩ, and the resistance of R8 is 200 KΩ. The function of the voltage measurement module is to divide the voltage, i.e. to divide the voltage through the resistors R7 and R8, so as to avoid the direct measurement of the voltage of the positive electrode +VBAT of the lithium battery BAT1, thereby preventing the voltage of the positive electrode +VBAT of the lithium battery BAT1 from being too high for measurement.

As shown in FIG. 6, the power supply input module includes a socket U2, which, in an example, is a USB socket; with the external power supply being a charging adapter, the socket U2 can be connected to the charging adapter through a charging cable. As shown in FIG. 1, the socket U2 corresponds to the socket 6 arranged on the body 4. A fuse F2 is connected between the socket U2 and the output terminal +5V_USB of the power supply input module. In an example, the fuse F2 has a rated voltage of 5 V and a rated current of 1 A. The output terminal +5V_USB of the power supply input module is connected to the VIN pin of the rechargeable battery module through a resistor R2, which, in one example, has a resistance of 3.9Ω.

As shown in FIG. 3, the output terminal +5V_USB of the power supply input module is directly connected to pin VDD50-I of the MCU module U1 to supply power to the MCU module U1. In addition, as shown in FIG. 7, the output terminal +5V_USB of the power supply input module is also connected to pin P46 of the MCU module U1 through the insertion detection module, so that the MCU module U1 can detect whether the power supply input module is connected to the external power supply. The power supply input module also includes capacitors C1 and C2. One terminal of the capacitor C1 is grounded, and the other terminal is connected to the output terminal +5V_USB; one terminal of the capacitor C2 is grounded, and the other terminal is connected to the output terminal +5V_USB. That is, the capacitors C1 and C2 are connected in parallel. In an example, the capacitance of the capacitor C1 is 100 nF, and the capacitance of the capacitor C2 is 10 μF.

As shown in FIG. 7, the insertion detection module includes a resistor R1, which has one terminal USB-DT connected to pin P46 of the MCU module U1 and the other terminal +5V_USB connected to the output terminal +5V_USB of the power supply input module; the resistor R1, as a pull-up current-limiting resistor, is used for +5V_USB to provide an input high-level signal to the I/O pin of the MCU module U1 through the resistor R1, so that the MCU module U1 can accurately detect the high level of 5 V.

According to the above circuit, the present invention provides the following method for indicating the power supply, so that the finger clip type pulse oximeter presents a visual result directly observable by the human eyes, and a user can more intuitively understand the power supply state of the finger clip type pulse oximeter.

The method for indicating the power supply comprises the following situations: When the socket U2 of the power supply input module is connected to the external power supply through the charging cable, the charging power supply reaches the network +5V_USB through the fuse F2, the +5V_USB power supply enters the MCU module U1 through the network USB-DT via the resistor R1, and pin P46 of the MCU module U1 will automatically detect that there is a high level of 5V, so it is considered that an external charging power supply is connected at this time;
if pin P15 of the MCU module U1 detects that the lithium battery BAT1 is in a charging state through the detection of the detection terminal LV_ADC, the MCU module U1 will send a pulse control signal of a certain frequency through pin P52, so that the first LED flashes in the first color (e.g. blue) at a predetermined frequency;
if pin P15 of the MCU module U1 detects that the lithium battery BAT1 is fully charged through the detection of the detection terminal LV_ADC when the external power supply is connected, the MCU module U1 will send a low-level signal through pin P52 to directly light the first LED, so that the first LED is always on in the first color (e.g. blue);
when the socket U2 of the power supply input module is not connected to the external power supply, pin P15 of the MCU module U1 will automatically detect the level of the network LV_ADC as soon as the finger clip type pulse oximeter is turned on; when the LV_ADC is in a low-voltage state identified by the program control algorithm (that is, the voltage of the detection terminal LV_ADC of the voltage measurement module is lower than a first predetermined voltage), it indicates that the lithium battery BAT1 starts to run low; at this time, the MCU module U1 will send a low-level signal through pin P49 to directly light the second LED, so that the second LED is always on in the second color (e.g. orange), thereby prompting the user to charge as soon as possible; and
when the detection terminal LV_ADC is in an extremely low-voltage state identified by the program control algorithm (that is, the voltage of the detection terminal LV_ADC of the voltage measurement module is lower than a second predetermined voltage, with the second predetermined voltage lower than the first predetermined voltage), it indicates that the lithium battery BAT1 runs seriously low; at this time, the MCU module U1 will send a pulse control signal of a certain frequency through pin P49, so that the second LED flashes in the second color (e.g. orange) at the predetermined frequency, thereby prompting the user that the oximeter is about to shut down automatically and please charge it immediately.

The above detailed description is only the description of the preferred examples of the present invention, and is not intended to limit the patent scope of the present invention. Therefore, all equivalent technical changes made by using the content of the present invention are included in the patent scope of the present invention.

1. A circuit, applied to a finger clip type pulse oximeter, for indicating a power supply by applying a double-color light-emitting diode (LED), characterized in that the circuit comprises:
an MCU module U1;
a double-color LED module, including a double-color LED D1 and connected to the MCU module U1;
a voltage measurement module connected to the MCU module U1;
a rechargeable battery module connected to the voltage measurement module; and
a power supply input module, which is suitable for connection with an external power supply and is connected to the MCU module U1 and the rechargeable battery module;
wherein the MCU module U1 is suitable for controlling the double-color LED D1 according to the states of the power supply input module and the rechargeable battery module, so that the finger clip type pulse oximeter presents a visual result directly observable by the human eyes.

2. The circuit for indicating a power supply by applying a double-color LED according to claim 1, characterized in that: with the power supply input module including a socket U2, a fuse F2 is connected between the socket U2 and the output terminal of the power supply input module; the output terminal of the power supply input module is connected to the rechargeable battery module, and connected to the MCU module U1 through an insertion detection module.

3. The circuit for indicating a power supply by applying a double-color LED according to claim 2, characterized in that: the insertion detection module includes a resistor R1, which has one terminal connected to the MCU module U1 and the other terminal connected to the output terminal of the power supply input module.

4. The circuit for indicating a power supply by applying a double-color LED according to claim 1, characterized in that: the rechargeable battery module comprises a lithium battery BAT1, and a charging management chip U3 connected to the output terminal of the power supply input module; the lithium battery BAT1, with its negative electrode grounded, has its positive electrode connected to the charging management chip U3 and connected to the MCU module U1 through the voltage measurement module.

5. The circuit for indicating a power supply by applying a double-color LED according to claim 4, characterized in that: the voltage measurement module includes resistors R7 and R8, one terminal of the resistor R7 being connected to the positive electrode of the lithium battery BAT1; the resistor R8 has its one terminal grounded, and the other terminal connected to the other terminal of the resistor R7 and then connected to the MCU module U1.

6. The circuit for indicating a power supply by applying a double-color LED according to claim 4, characterized in that: the double-color LED D1 comprises a first LED capable of exhibiting a first color, and a second LED capable of exhibiting a second color, with the first color different from the second color; the double-color LED module further comprises resistors R4 and R5; the anode of the first LED is connected to a VDD pin of the MCU module U1 through the resistor R4, and the anode of the second LED is connected to the same VDD pin of the MCU module U1 through the resistor R5; the cathode of the first LED is directly connected to one I/O pin of the MCU module U1, and the cathode of the second LED is directly connected to the other I/O pin of the MCU module U1.

7. The circuit for indicating a power supply by applying a double-color LED according to claim 6, characterized in that:
the above description that “the finger clip type pulse oximeter presents a visual result directly observable by the human eyes” is specifically as follows:
when the power supply input module is connected to the external power supply, and the lithium battery BAT1 is being charged, the MCU module U1 controls the first LED to flash in the first color at a predetermined frequency;
when the power supply input module is connected to the external power supply, and the lithium battery BAT1 is fully charged, the MCU module U1 controls the first LED to be always on in the first color;
when the power supply input module is not connected to the external power supply, and the MCU module U1 detects that the voltage of the detection terminal of the voltage measurement module is lower than a first predetermined voltage, the MCU module U1 controls the second LED to be always on in the second color; and
when the power supply input module is not connected to the external power supply, and the MCU module U1 detects that the voltage of the detection terminal of the voltage measurement module is lower than a second predetermined voltage, the MCU module U1 controls the second LED to flash in the second color at a predetermined frequency;
wherein the second predetermined voltage is lower than the first predetermined voltage.

8. The circuit for indicating a power supply by applying a double-color LED according to claim 4, characterized in that: the charging management chip has a VIN pin connected to the power supply input module, a BAT pin connected to the lithium battery BAT1 for charging current output, and a CHG pin connected to an indication LED D2 for charging state output, the indication LED D2 capable of exhibiting a third color.

9. A method, applied to a finger clip type pulse oximeter, for indicating a power supply by applying the circuit according to claim 1, characterized in that the method is specifically as follows:
when the power supply input module is connected to the external power supply, and the lithium battery BAT1 of the rechargeable battery module is being charged, the MCU module U1 controls the first LED of the double-color LED D1 to flash in the first color at a predetermined frequency;
when the power supply input module is connected to the external power supply, and the lithium battery BAT1 of the rechargeable battery module is fully charged, the MCU module U1 controls the first LED of the double-color LED D1 to be always on in the first color;
when the power supply input module is not connected to the external power supply, and the MCU module U1 detects that the voltage of the detection terminal of the voltage measurement module is lower than a first predetermined voltage, the MCU module U1 controls the second LED of the double-color LED D1 to be always on in the second color; and
when the power supply input module is not connected to the external power supply, and the MCU module U1 detects that the voltage of the detection terminal of the voltage measurement module is lower than a second predetermined voltage, the MCU module U1 controls the second LED of the double-color LED D1 to flash in the second color at a predetermined frequency;
wherein the second predetermined voltage is lower than the first predetermined voltage.

10. An oximeter for indicating a power supply by applying a double-color LED, characterized in that: the oximeter, being of a finger clip type pulse oximeter, comprises a body and a circuit according to claim 1; the body is provided on the surface with a display screen and an indication position located on one side of the display screen, with the double-color LED D1 set corresponding to the indication position.
